(19) United States
(12) Patent Application Publication
(10) Pub. No.: US 2016/0284080 A1
(43) Pub. Date: Sep. 29, 2016

(54) VASCULATURE MODELING

(71) Applicant: Sabanci University, Tuzla - Instanbul (TR)

(72) Inventors: Gozde Unal, Istanbul (TR); Suheyla Cetin, Istanbul (TR)

(21) Appl. No.: 14/671,008

(22) Filed: Mar. 27, 2015

(51) Int. Cl. G06T 7/00; G06T 17/20; G06T 5/00; A61B 5/02

(52) U.S. Cl. CPC G06T 7/0012; A61B 5/02007; G06T 17/20; G06T 5/003; G06T 7/0051; G06T 7/0079; G06T 2207/30101

(57) ABSTRACT

The present invention relates to modeling of vascular structures, and in particular to extracting a vessel tree from medical-images of vascular anatomy. A respective method comprises among others the steps of providing an image of at least one vessel, obtaining multiple measurements from the image for a first point of the image, and fitting a four-dimensional tensor to the measurements. Based on said four-dimensional tensor fitted to the measurements, a vessel direction in a vessel tree is determined and a model of the vascular structures is generated based on at least the determined vessel direction.

### 1. FIELD OF THE INVENTION

The present invention relates to modeling of vascular structures, and in particular to extracting a vessel tree from medical images of vascular anatomy.

### 2. TECHNICAL BACKGROUND

Vascular problems of human bodies can cause deadly medical events such as heart attacks and strokes. The buildup of plaque inside a vascular system, for example, can lead to strokes or coronary heart disease. One important step for detecting and analyzing vessel anomalies and pathologies such as aneurysms, stenosis and plaques involves the extraction of vascular structures such as coronary and cerebral arteries.

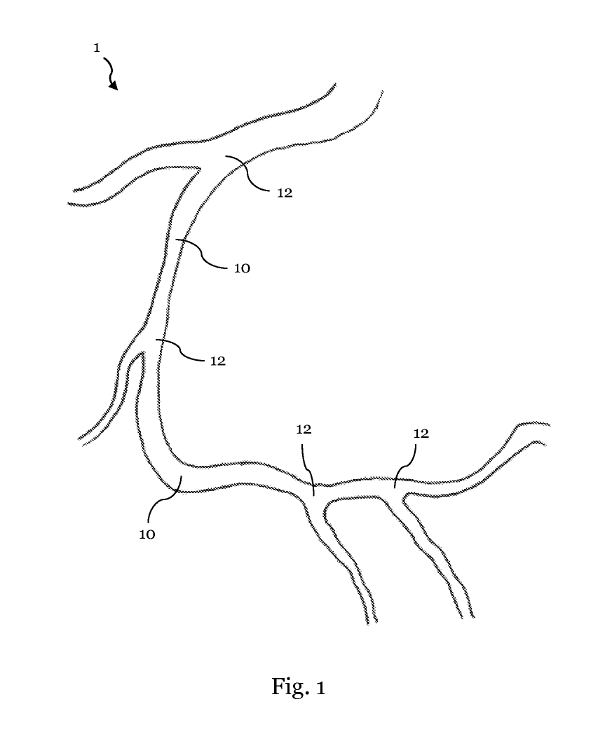

FIG. 1 illustrates a schematic portion of an exemplary vasculature 1. The illustrated vasculature 1, or vascular structure 1, can for example be part of an arterial or venous system. As can be seen in FIG. 1, the vasculature 1 having vessels 10 can feature furcations, such as the illustrated bifurcations 12. It is known that vascular structures can also have more complex branching, such as trifurcations, pentafurcations or septafurcations. In general, n-furcations are locations where n branches exit from the same vessel point. It is in particular these n-furcations, which have to be modeled properly.

Blood vessels can be imaged by means of angiography, which provides a powerful technique for identifying and tracking vascular diseases. Angiography is typically performed using computer tomography (CT), magnetic resonance imaging (MRI), or the like. Thereby two- or three-dimensional angiographic data or images can be obtained.

The manual segmentation of vascular structures is an exhaustive task. Therefore, computer-based segmentation algorithms have been developed to extract patient-specific vasculature models from imaging data, for example from angiographic images. With such computer-based segmentation the vasculature can be differentiated from non-vascular structures, background or even noise in an automated manner. However, the presence of branchings in vessels, such as the bifurcations 12 illustrated in FIG. 1, can disturb the automated creation of patient-specific vessels. Similarly, also a variation in tubular radii can bring additional challenges.

A method for vasculature segmentation is proposed in the article “Vessel Tractography Using an Intensity based Tensor Model with Branch Detection” of S. Cetin et al., published 2013 in the IEEE Transactions on Medical Imaging (vol. 32, issue 2). Therein described is a technique using second order tensors for modeling the vasculature. With an initial segmentation step a 3D segmented volume is produced, followed by a separate branch detection scheme.

However, there still exists a need for a fast and robust vasculature segmentation technique.

### 3. SUMMARY OF THE INVENTION

According to one aspect of the invention, a method is disclosed for modeling vascular structures. Accordingly, a model of vasculature structures is obtained, or in other words, the method provides a technique for performing vasculature modeling. Preferably, according to the present invention, a vessel tree is extracted from medical images of vascular anatomy.

An image of at least one vessel is provided. Preferably, said image is a medical image of vascular anatomy, an angiographic image or a vascular image obtained with any vascular imaging modality. Multiple measurements from this image are obtained for a first point P₁ of the image, which measurements are preferably directional measurements. It will be appreciated that the number of measurements obtained for a point of said image depends on the image quality, as well as on the complexity of the furcations present in the image. As an example, 64 measurements or directional measurements are obtained for the first point P₁, i.e. 64 measurements with different orientations are obtained for the first point P₁. In another example, 128 or 256 measurements or directional measurements are obtained for the first point P₁. Preferably, the first point P₁ is located on the at least one vessel of the provided image. Accordingly, the measurements are preferably obtained for a point which is located on the at least one vessel. Further preferred the first point P₁ is located on a centerline of the at least one vessel. Thus, a centerline of a vessel is tracked with the present invention.

A four-dimensional tensor is fitted to the measurements. Preferably, the tensor is fitted holistically to the obtained multiple measurements. A vessel direction is determined based on said four-dimensional tensor fitted to the measurements. Preferably, a number of existing vessel directions present at the first point P₁ is thereby determined. A model of the vascular structure is generated based on at least the determined vessel direction or preferably based on the determined vessel directions, if more than one vessel direction is determined (e.g. at a vessel furcation). The determined vessel direction is denoted as ĝ herein.

The image can be obtained by any suitably means, such as for example by means of MRI, CT, or the like. Thus, in a preferred embodiment, the providing the image comprises obtaining the image by means of an image modality, which modality is preferably suited for imaging vascular anatomy. Preferably, the image is a three-dimensional image and the measurements are formed in three dimensions, such that extensive information on the vessel structure can be obtained. Preferably, the measurements are formed on a unit 2-sphere in three dimensions. Preferably the measurements are formed at the first point P₁, further preferred for multiple orientations. It will be appreciated that the multiple orientations may correspond to the multiple measurements. The measurements or measured values provide information about the first point P₁ of the image, and can for example provide information about intensity values at this first point P₁. By using a four-dimensional tensor for modeling vascular structures, it is possible to obtain a vascular structure that can model asymmetric junction scenarios, such as they are for example present in a bifurcation. Accordingly, n-furcations of vascular structures are modeled using a tensor, preferably a higher-order tensor, embedded in 4D. It is thus advantageously possible to model both antipodal asymmetry and the symmetry of a vessel n-furcation in an accurate and very fast manner, with a computation time per dataset or image of less than 30 seconds.

Preferably, the four-dimensional tensor is a higher-order tensor, which is further preferred of third or fourth order. With such higher-order tensors branching points can be naturally modeled. Accordingly, using higher-order tensor embedded in four dimensions, it is possible to extract whole vascular trees in an efficient manner. It will be appreciated that the four-dimensional tensor can be of even higher order, such as e.g. of fifth or sixth order.

Preferably the three dimensions of the four-dimensional tensor define an orientation vector on a unit-2 sphere. In another preferred embodiment, three dimensions of the four-dimensional tensor are the three dimensions of an orientation vector on a unit 2-sphere. Preferably a fourth dimension of the four-dimensional space, on which the tensor is constructed, is defined as a sharpening function, which sharpening function is preferably a sigmoid function. This utilization of a sigmoid function can provide a high pass filter on the measurements by sharpening the measurements. Further preferred, an argument of the sharpening function, such as e.g. an argument of the sigmoid function, includes at least one of the measurements, or the respective measurements. Similarly, in another preferred embodiment, the sharpening function is a Gompertz function. Further preferred, an argument of said Gompertz function includes at least one of the measurement, or the respective measurements. In a general manner, any function providing suppression of measurements when the image is not symmetric along the sampled measurement is suitable.

In a preferred embodiment, the obtaining of the measurements comprises that intensity measurements from inside a cylinder are calculated, which cylinder is having an axis passing through the first point P₁. Preferably, the obtaining of the measurements further comprises that an intensity value of a sphere placed inside the cylinder and an intensity value of the rest of the cylinder are calculated. Further preferred, the sphere is spanned around the first point P₁. Preferably, the step of obtaining the measurements comprises squaring a difference of the intensity value of the sphere and the intensity value of the rest of the cylinder. Accordingly, with this intensity-based method a vessel can be traced in a fast manner.

Preferably the obtaining the measurements comprises that image gradient magnitudes are calculated on a side surface of a circular cylinder. This calculating the image gradient magnitudes preferably comprises calculating an image gradient vector on a circle point fₖ being located on the side surface of the circular cylinder, which circular cylinder is preferably having an axis passing through the first point P₁. Preferably, the first point P1 is located on a centerline of the at least one vessel, such that the axis of the circular cylinder is preferably passing through a respective centerline point. Further preferred, the calculating the image gradient magnitudes comprises calculating a scalar product of the image gradient vector and a unit vector pointing from the circle point fₖ to the axis of the circular cylinder. Further preferred the unit vector is perpendicular to the axis of the circular cylinder. The resulting measurements are advantageously less sensitive to noise due to this flux-based measurement model. Further preferred the obtaining the measurements comprises accumulating the image gradient magnitudes.

Preferably the four-dimensional tensor is fitted to the measurements utilizing an estimation technique, which is preferably a least-squares technique. Thereby a suitable four-dimensional tensor can be estimated from the measurements in a fast and accurate manner. Likewise the tensor components of said four-dimensional tensor can be estimated via a least-squares estimator. It will be appreciated that also other estimation techniques may be applied, such as e.g. non-linear estimation techniques.

Preferably the vessel direction is determined by decomposing the four-dimensional tensor, which is fitted to the measurements, into its components. Thus, the determining the vessel direction preferably comprises decomposing the four-dimensional tensor into its components. For the purpose of this decomposition, a so-called Tucker decomposition is preferably utilized. Alternatively or additionally, the four-dimensional tensor is decomposed into its components by decomposing the four-dimensional tensor into at least one rank-1 term. Thereby the four-dimensional tensor representation is decomposed into an optional isotropic part, at least one rank-1 terms representing at least one individual fiber peak, as well as a small residual covering among others noise. With these techniques the four-dimensional tensor can be efficiently evaluated for vascular n-furcation modeling. The rank-1 approximation method thereby further reduces the computation time for modeling vascular structures in the sense of the present invention, while the Tucker decomposition proved to be even faster in the context of the present invention. It will be appreciated that several vessel directions may be determined for a given point, for example if a bifurcation is present at said given point.

In another preferred embodiment the method comprises the steps of selecting an initial seed point and estimating a vessel radius of the selected initial seed point. Preferably, the initiate the seed point is located on the at least one vessel. Accordingly, the extraction of the vessel tree starts from this initial seed point. The selection is preferably done by an operator or user, while the following tracing of the vessel is performed in a fast and automated manner. Accordingly, the inventive method is not only fast, but also provides an easy user interaction with a single seed selection. The initial seed selection can also be automated by further pre-processing and segmentation, such as by segmenting the known anatomy in the heart (e.g. ostium) for coronary arteries, or using a brain atlas to locate a seed point for the cerebral arteries.

In a further preferred embodiment the method comprises after the determining the vessel direction that a vessel thickness is estimated along the vessel direction. Preferably, this estimation of the vessel thickness is performed along the determined vessel direction. Preferably, said estimating is based on a geometrical model applied to the measurements, which model is further preferred a cylindrical, conical, and/or spherical model. Preferably, said estimation of the vessel thickness comprises that image gradient magnitudes are accumulated. The image gradient magnitudes are in turn preferably calculated on a surface or a side surface of a cylinder having in axis coinciding with the determined vessel direction. Accordingly, a cylinder is aligned such that it points into the direction of the vessel direction which was determined in advance. Preferably, the radius of the cylinder is varied for estimating an optimal vessel thickness. By utilizing such a simple maximum norm criterion of cylindrical measurements over a range of radii, the thickness of the vessel lumen can be estimated fast and reliable. Further preferred, the generating the model of the vessel structure is further based on in the vessel thickness. Accordingly, an informative model of the vascular structure can be obtained, whereby both tubular sections and n-furcations of the vascularities are modeled in a simultaneous manner. By determining both the vessel direction, i.e. vessel centerline, and thickness (or radius) of the vessel, a surface of the vessel can easily be defined and incorporated into the modeling of the vessel tree.

Preferably the method further comprises after the determining the vessel direction or generation of the model the step of advancing to a second point P₂ along the vessel direction. Afterwards, one or more of the above-outlined steps are repeated for the second point P₂. Accordingly, the method features an iterative algorithm for tracing the vessel in an automated manner.

Preferably, with determining the vessel direction based on the four-dimensional tensor fitted to the measurements, at least three vessel directions are determined. In a particularly preferred embodiment the determining the vessel direction comprises detecting a branching of the at least one vessel at the first point P₁ based on the four-dimensional tensor fitted to the measurements, and in particular based on the at least three vessel directions. Preferably, said branching is a bi-, tri- or generally an n-furcation of said at least one vessel. Accordingly, a number of principal directions of the vessel may preferably be estimated for the first point P₁, wherein said first point P1 is preferably located on said vessel. The utilization of a four-dimensional tensor thereby allows for efficiently estimating such vessel branches, as it allows for modeling both symmetric and asymmetric situations. Preferably, said detection of a branching may comprise detecting several vessel directions for the first point P₁, or detecting several principal directions for said first point P₁. Based on the several directions, a vessel branching, such as for example a bifurcation, can be distinguished. Further preferred, the generating the model of the vascular structures is further based on said branching, i.e. the detected vessel branch.

According to another aspect of the invention an apparatus for modeling vascular structures is disclosed. A means is provided for providing an image of at least one vessel. A means is provided for obtaining multiple measurements from the image for a first point P₁ of the image. A means is provided for fitting a four-dimensional tensor to the measurements. A means is provided for determining a vessel direction based on the four-dimensional tensor fitted to the measurements. A means is provided for generating a model of the vascular structures based on at least the vessel direction.

Another aspect of the disclosure provides and an apparatus comprising a processor and a memory storing a program and for memorizing data that is processed by the processor. The processor is thereby configured with the memory and the program to cause the apparatus at least to provide an image of at least one vessel. The processor further causes the apparatus to obtain multiple measurements from the image for a first point P₁ of the image. The processor further causes the apparatus to fit a four-dimensional tensor to the measurements. Further, the processor causes the apparatus to determine a vessel direction based on the four-dimensional tensor fitted to the measurements. Further the processor causes the apparatus to generate a model of the vascular structures based on at least the vessel direction.

Another aspect of the disclosure provides a computer-readable medium comprising instructions to perform the steps of the inventive method described herein when executed on a computer.

### 4. DESCRIPTION OF PREFERRED EMBODIMENTS

In the following various aspects of the present invention are described more fully with reference to the accompanying drawings. The drawings are only for the purpose of illustrating preferred embodiments and are not to be construed as limiting the invention.

FIG. 1 illustrates a schematic representation of a vascular structure having several bifurcations;

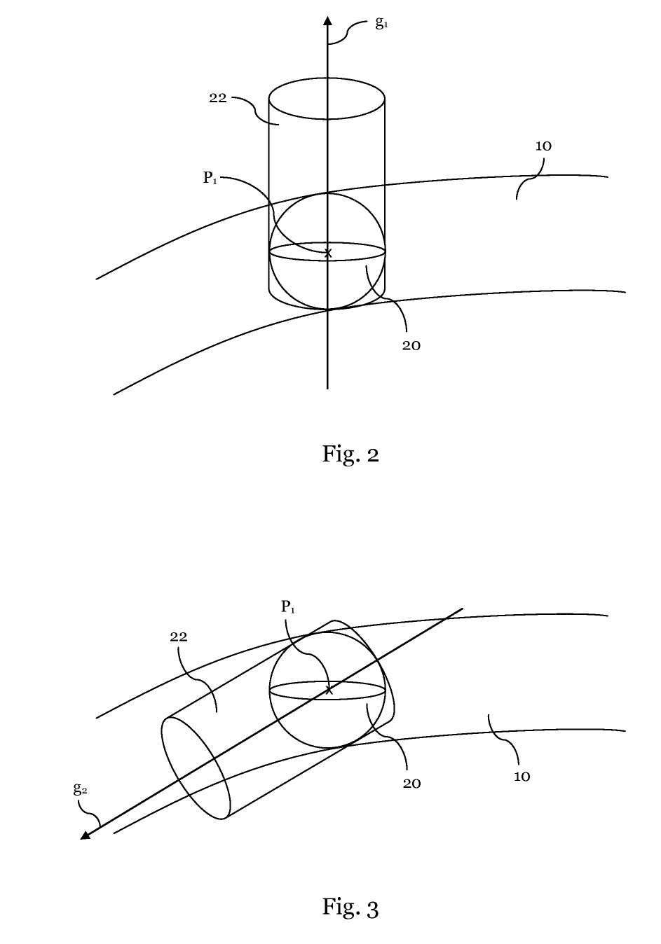

FIGS. 2 and 3 illustrate schematically a method for obtaining a directional measurement according to one embodiment;

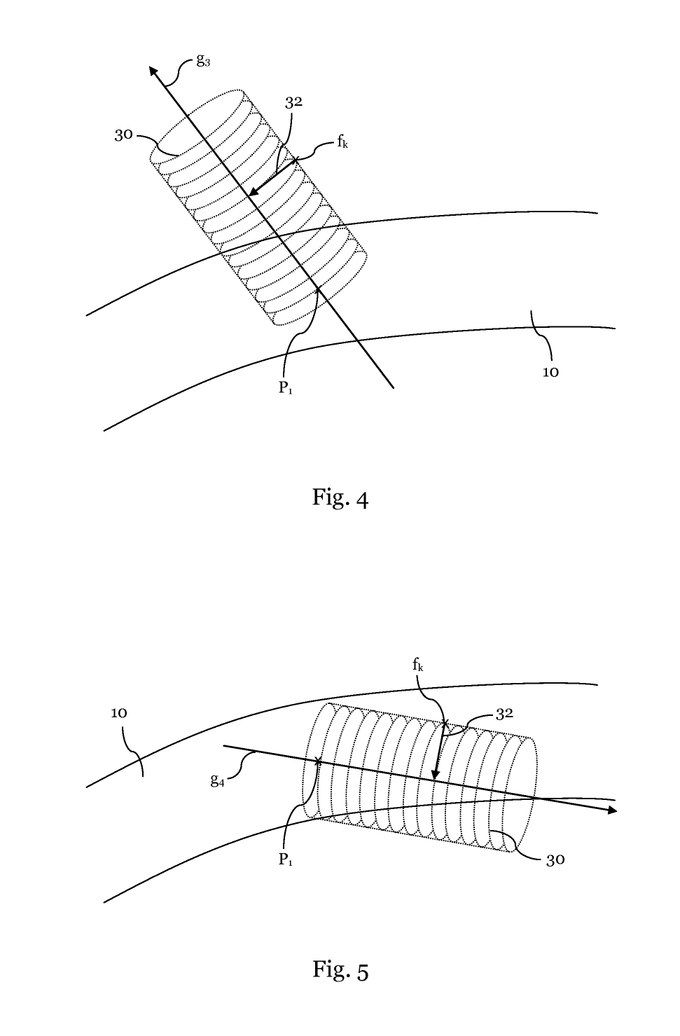

FIGS. 4 and 5 illustrate schematically a method for obtaining a directional measurement according to another embodiment;

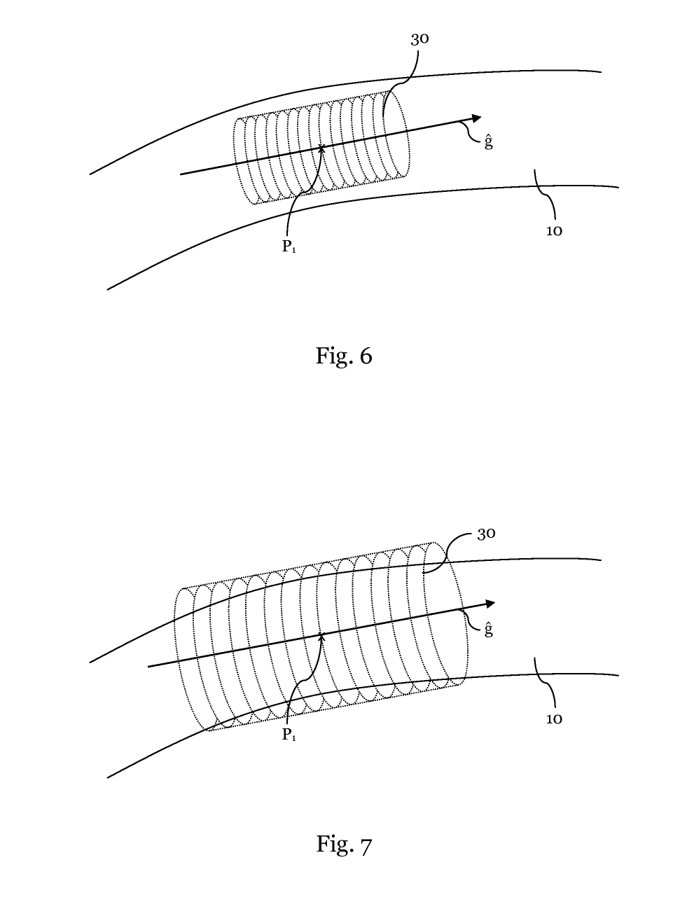

FIGS. 6 and 7 illustrate schematically a method for estimating a vessel thickness according to a further embodiment;

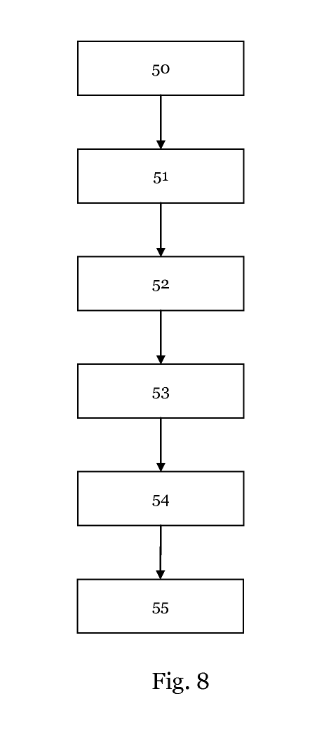

FIG. 8 illustrates an exemplary method for modeling vascular structures according to another embodiment;

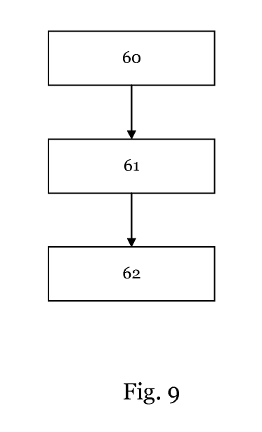

FIG. 9 illustrates an exemplary method for modeling of vascular structures according to another embodiment;

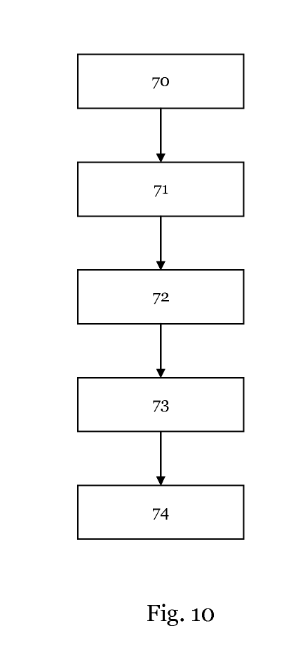

FIG. 10 illustrates an exemplary method for modeling vascular structures according to another embodiment, and

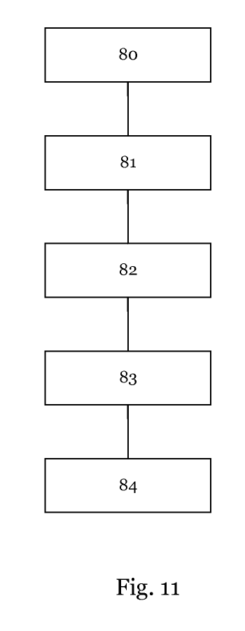

FIG. 11 illustrates a functional block diagram of an exemplary apparatus.

With reference to FIGS. 2 and 3, an exemplary method for obtaining a directional measurement from image intensities at various spatial directions from a first point of an image, such as e.g. of an angiographic image, is described in the following. As can be seen, a first point P₁ is located on a vessel 10. Along a certain direction or along an orientation vector gᵢ (which is preferably defined on a unit-2 sphere) a cylinder 22 is aligned. The cylindrical axis of the cylinder 22 thereby coincides with the orientation vector g₁ (or g₂ in FIG. 3) and the first point P₁. Further, a sphere 20 centered around the first point P₁ is illustrated. Preferably, the thickness of the sphere 20 and the cylinder 22 are approximately equal to the thickness of vessel 10.

For obtaining a measurement according to this embodiment, an intensity mean value s(sph) of the sphere 20 as well as an intensity mean value s(cyl) of the rest of the cylinder 22 are calculated. Afterwards a difference between the intensity mean s(sph) of the sphere 20 and the intensity mean s(cyl) of the rest of the cylinder S2 are obtained, and the difference is squared afterwards.

Such intensity measurements are computed along several or multiple orientation vectors gᵢ. As the intensity values of the vessel 10 are typically different than those of the non-vessel parts of the image, it is reasonable that the measurements resulting from FIGS. 2 and 3 are different.

With reference to FIGS. 4 and 5, a flux-based measurement model is described in the following for obtaining directional measurements. Again, a first point P₁ is located on a vessel 10. The model for obtaining a measurement consists of a hollow cylinder 30 aligned along a sampled orientation gᵢ. The cylinder 30 is represented by a stack of circles, which are equi-angularly discretized into numerous points in polar coordinates. Accordingly, the points are provided on the side surface of the cylinder 30.

Next, when sampling a set of orientation vectors gᵢ over the unit-2 sphere, each measurement at a given direction in gᵢ is modelled as a flux-based feature, which is created by an accumulation of image gradient magnitudes. Thereby, for each sampled point, such as point fₖ, an image gradient vector is obtained. Afterwards, a scalar product of this image gradient vector and a vector 32, pointing from said sampled point fₖ towards the axis of the hollow cylinder 30, is computed. Accordingly, by computing this scalar product, only those image gradient vectors are further considered which are not perpendicular to the vector 32 pointing from the sampled point fₖ towards the axis. Thus, if the axis is aligned along the true vessel direction, the image gradient vector at point fₖ is parallel to the vector 32, and the resulting scalar product is maximal. Preferably, diametrically opposed points on the hollow cylinder 30 can be paired for minimizing the effects of bright structures which may be situated near the vessels.

In this way, image gradient vectors are computed for all discretized points of each circle of the hollow cylinder 30. The accumulation of these image gradient magnitudes then results in the measurement. Again, such a flux-based measurement is computed for each sampled direction vector gᵢ. As an example, two different direction vectors g₃ and g₄ are illustrated in FIG. 4 and FIG. 5, respectively.

With reference to FIGS. 6 and 7, an exemplary method for estimating a thickness of the vessel 10 is described next. As the thickness of the vessel 10 is directly related to the radius of the cylinders 30, the obtained measurements become lower when the diameter of the cylinder is below or above the true or the actual vessel lumen thickness. Accordingly, the measurement is the largest when in the cylinder 30 is tangent and along the direction of the vessel 10, as the calculated scalar products are maximal in this situation (cf. the above discussion). According to the present invention, the hollow cylinders 30 are similarly construed as in FIGS. 4 and 5, however, they are aligned along the determined, i.e. modeled vessel direction ĝ. The measurements are then estimated for multiple radii, and the best radius is selected.

FIG. 8 illustrates an exemplary method for modeling vascular structures. In step 50, as an initialization, a user-defined single seed point is selected. At this seed point a radius-estimation is performed for estimating the radius at this seed point, preferably by using a maximum tensor norm criterion as known in the art. In step 51 a calculation of measurements for each centerline direction gᵢ, with cylinders having in radius r, are obtained, i.e. vessel measurements are created. Said calculation of directional measurements can be performed by calculating intensity values and/or by calculating image gradients. In step 52 four-dimensional higher-order tensors are fitted to the measurements, i.e. a four-dimensional higher order tensor modeling is performed. In step 53 an estimation of each principle direction is performed, such that vessel directions are extracted. In step 54 higher-order tensor tractography is performed, as it is advanced to the next centerline point based on the detected vessel directions. In step 55, as a last step, a radius is estimated along the estimated direction or along the vessel orientation.

In a preferred embodiment, S(g) is a measurement, g=[g¹, g², g³] is an orientation vector on the unit-2 sphere, and g⁴ is defined with a sigmoid function, with S(g) included in the argument of said sigmoid function. Utilizing this model in four dimensions lifts the limitation with symmetry of measurements. The sigmoid function is designed as a high pass filter on the measurements by sharpening the measurement along the highly varying directions since the absolute intensity variation along the vessel and the branches are expected to be more than the variation in other directions. The resulting four-dimensional vector p=[g¹, g², g³, g⁴] lies on the unit-3 sphere, which carry the tensor modeling to four-dimensional space. The four-dimensional space constructed on the 3-sphere is achieved by the g⁴ coordinates. It will be appreciated that also other forms of g⁴ would work as well, as long as they provide suppression of measurements when the image is not symmetrical along g.

For testing the inventive technique, the proposed algorithm was implemented in Matlab and C. Following experiments were run on an Intel processor Xeon X560 (2.67 GHz) CPU computer with 64 GB memory. The proposed algorithm needed less than 30 seconds for creating the whole vessel tree in the CTA data experiments of the Rotterdam evaluation framework. For an MRA experiment of cerebral vasculature extraction, the whole vessel tree is captured in about 60 seconds using a Tucker decomposition method for analyzing the four-dimensional tensors. Further, time is doubled using the rank-1 approximation method. Improvements in scores of the proposed method over previous ones can also be observed in all measures including overlap and accuracy. Moreover, the proposed algorithm proved to be the fastest among all participating methods of the Rotterdam Coronary Artery Algorithm Evaluation Framework. In addition, when comparing the proposed algorithm with previous methods, it is found that the proposed method is capable of finding more number of branches. Improvement in the performance of the vessel tractography approach could be observed in both applications to the coronary arteries and the cerebral arteries. It will be appreciated that the proposed vessel tractography method can also easily be adapted to other modalities, such as for example for the extraction of cerebral veins from venography image volumes.

With reference to FIG. 9, there is provided a method for modeling of vascular structures. In step 60 a principle direction ĝ of a vessel is determined for a first point P₁. At step 61 it is advanced along the determined principal direction ĝ towards a next centerline point P₂. In step 62, a radius-estimation along the determined vessel orientation is conducted. Afterwards, the method is continued at step 60 until the entire vessel tree is modelled, an interruption of the vessel structure is determined, or a user terminates the modeling.

The skilled person understands that a number of vessel directions can be determined, if the sampled centerline point is located on e.g. a vessel furcation. For example, several principal directions can be extracted from the four-dimensional higher-order tensor. According to the present invention, each of the determined vessel directions can be subsequently tracked for extracting the whole vessel tree.

With reference to FIG. 10 a method for modeling vascular structures is described in the following. In step 70 an image of at least one vessel is provided, such as e.g. an angiographic image or a medical image of vascular anatomy. In step 71, multiple measurements from the image are obtained for a first point P₁ of the image. The skilled person understands that, in the sense of the present invention, the number of necessary measurements depends on the resolution of the images, as well as on the complexity of the furcations. As an example, 64, 128 or 256 measurements are obtained for each sampled point, such as e.g. for the first point P₁. It will be appreciated, that also other number of measurements can be obtained. At step 72 a four-dimensional tensor is fitted to the multiple measurements. At step 73 a vessel direction ĝ is determined based on the four-dimensional tensor fitted to the measurements. Preferably this determination comprises an estimation of whether a vessel branch is present at said point P₁. More than two detected directions correspond to a bifurcation, more than three directions correspond to a trifurcation, and so on. At step 74 a model of the vascular structures is generated based on at least the vessel direction ĝ.

With reference to FIG. 11 an apparatus according to the present invention for modeling vascular structures is described in the following. The apparatus comprises means 80 for providing an image of at least one vessel, such as e.g. an angiographic image or a medical image of vascular anatomy. The apparatus further comprises means 81 for obtaining multiple measurements from the image for a first point P₁ of the image. Further, the apparatus comprises means 82 for fitting a four-dimensional tensor to the measurements. The apparatus also comprises means 83 for determining a vessel direction ĝ based on the four-dimensional tensor fitted to the measurements. In addition, the apparatus comprises means 84 for generating a model of the vascular structures based on at least the vessel direction ĝ.

It will be appreciated that the functions described herein may be implemented in hardware or software or any combination thereof. If implemented in software, the functions may be stored as one or more instructions on a computer-readable medium. Further, the various operations of methods described above may be performed by any suitably means capable of performing the operations, such as various hardware and/or software components, circuits and/or modules. The modules may correspond to one or more processors and/or memory devices. Further, all functions and techniques described herein may be combined and/or performed in different order, depending on the individual application.

1. A method for modeling vascular structures, the method comprising:
a) Providing an image of at least one vessel;
b) Obtaining multiple measurements from the image for a first point P₁ of the image;
c) Fitting a four-dimensional tensor to the measurements;
d) Determining a vessel direction (ĝ) based on the four-dimensional tensor fitted to the measurements, and
e) Generating a model of the vascular structures based on at least the vessel direction (ĝ).

2. The method of claim 1, wherein the first point P₁ is located on the at least one vessel, and preferably on a centerline of the at least one vessel.

3. The method of claim 1, wherein the four-dimensional tensor is a higher-order tensor of preferably third or fourth order.

4. The method of claim 1, wherein three dimensions of the four-dimensional tensor define an orientation vector on a unit 2-sphere.

5. The method of claim 1, wherein a fourth dimension of the four-dimensional tensor is defined as a sharpening function, which sharpening functions is preferably a sigmoid function or a Gompertz function.

6. The method of claim 5, wherein an argument of the sharpening function includes at least one of the measurements.

7. The method of claim 1, wherein the image is a three-dimensional image and wherein the measurements are formed at the first point P₁, preferably for multiple orientations, and further preferred on a unit 2-sphere in three dimensions.

8. The method of claim 1, wherein the obtaining the measurements comprises calculating intensity measurements from inside a cylinder having an axis passing through the first point P₁.

9. The method of claim 8, wherein the obtaining the measurements comprises squaring a difference of an intensity value of a sphere placed inside the cylinder and an intensity value of the rest of the cylinder.

10. The method of claim 1, wherein the obtaining the measurements comprises calculating image gradient magnitudes on a side surface of a circular cylinder.

11. The method of claim 10, wherein the calculating the image gradient magnitudes comprises calculating an image gradient vector at a circle point fₖ being on the side surface of the circular cylinder, which circular cylinder preferably has an axis passing through the first point P₁.

12. The method of claim 11, wherein the calculating the image gradient magnitudes comprises calculating a scalar product of the image gradient vector and a unit vector pointing from the circle point fₖ to the axis of the circular cylinder.

13. The method of claim 12, wherein the unit vector is perpendicular to the axis of the circular cylinder.

14. The method of claim 10, wherein the obtaining the measurements comprises accumulating the image gradient magnitudes.

15. The method of claim 1, wherein the four-dimensional tensor is fitted to the measurements utilizing an estimation technique, which estimation technique is preferably least-squares technique.

16. The method of claim 1, wherein the vessel direction is determined by decomposing the four-dimensional tensor fitted to the measurements into its components.

17. The method of claim 16, wherein the four-dimensional tensor is decomposed into its components utilizing a Tucker decomposition.

18. The method of claim 16, wherein the four-dimensional tensor is decomposed into its components by decomposing the four-dimensional tensor into at least one rank-1 term.

19. The method of claim 1, further comprising:
selecting an initial seed point, and
estimating a vessel radius of the selected initial seed point.

20. The method of claim 19, wherein the initial seed point is located on the at least one vessel.

21. The method of claim 1, further comprising after step d):
estimating a vessel thickness along the vessel direction (ĝ), said estimating being preferably performed along the vessel direction (ĝ).

22. The method of claim 21, wherein the estimating the vessel thickness is based on a geometrical model applied to the measurements, and preferably comprises accumulating image gradient magnitudes calculated on a surface of a cylinder having an axis coinciding with the vessel direction (ĝ).

23. The method of claim 21, wherein the generating the model of the vascular structures is further based on the vessel thickness.

24. The method of claim 1, further comprising after step d) or step e):
advancing to a second point P₂ along the vessel direction (ĝ) and repeating at least steps b)-d) for the second point P₂.

25. The method of claim 1, wherein at least three vessel directions are determined with determining the vessel direction (ĝ) based on the four-dimensional tensor fitted to the measurements, and wherein the determining the vessel direction (ĝ) comprises detecting a branching of the at least one vessel at the first point P₁ based on the at least three vessel directions, and wherein the generating the model of the vascular structures is further based on said branching, which branching is preferably a bi-, tri- or n-furcation of the at least one vessel.

26. An apparatus for modeling vascular structures, the apparatus comprising:
means for providing an image of at least one vessel;
means for obtaining multiple measurements from the image for a first point P₁ of the image;
means for fitting a four-dimensional tensor to the measurements;
means for determining a vessel direction (ĝ) based on the four-dimensional tensor fitted to the measurements, and
means for generating a model of the vascular structures based on at least the vessel direction (ĝ).

27. An apparatus, comprising a processor and a memory storing a program and for memorizing data that is processed by the processor, wherein the processor is configured with the memory and the program to cause the apparatus at least:
to provide an image of at least one vessel;
to obtain multiple measurements from the image for a first point P₁ of the image;
to fit a four-dimensional tensor to the measurements;
to determine a vessel direction (ĝ) based on the four-dimensional tensor fitted to the measurements, and
to generate a model of the vascular structures based on at least the vessel direction (ĝ).

28. A computer-readable non-transitory medium comprising instructions to perform the steps of claim 1 when executed on a computer.
